SEQUENCE LISTING (1 sequence)

SEQ ID NO: 1 - protein, 273 aa, Artificial Sequence
  Chemically Synthesized Fusion Protein
MALPKTGKPTAKQVVDWAINLIGSGVDVDGYYGRQCWDLPNYIFNRYWNFKTPGNARDMAWYRYPEGFKVFRNTSDFVPKPGDIAVWTGGNYNWNTWGHTGIVVGPSTKSYFYSVDQNWNNSNSYVGSPAAKIKHSYFGVTHFVRPAYKAEPKPTPPLESTPATRPVTGSWKKNQYGTWYKPENATFVNGNQPIVTRIGSPFLNAPVGGNLPAGATIVYDEVCIQAGHIWIGYNAYNGNRVYCPVRTCQGVPPNQIPGVAWGVFKVEHHHHHH

(19) United States
(12) Patent Application Publication
(10) Pub. No.: US 2015/0335719 A1
(43) Pub. Date: Nov. 26, 2015

(54) Intravitreal Injection of a Chimeric Phage Endolysin Ply187; Protection from Staphylococcus aureus Endophthalmitis

(71) Applicants: The United States of America, as Represented by the Secretary of Agriculture, Washington, DC (US); Wayne State University, Detroit, MI (US)

(72) Inventors: David M. Donovan, Baltimore, MD (US); Pawan Kumar Singh, Detroit, MI (US); Ashok Kumar, Troy, MI (US)

(21) Appl. No.: 14/286,830

(22) Filed: May 23, 2014

(51) Int. Cl. A61K 38/48; A61K 47/48

(52) U.S. Cl. CPC A61K 38/48; A61K 47/48715; C12Y 304/00

(57) ABSTRACT

The treatment of endophthalmitis is challenging due to the emergence of MDR bacteria. We evaluated the therapeutic potential of Ply187AN-KSH3b, a chimeric phage endolysin derived from the Ply187 prophage in a mouse model of Staphylococcus aureus endophthalmitis. The endolysin was injected intravitreally in C57BL/6 mouse eyes at 6 h and 12 h post S. aureus infection. The disease progression was monitored by ophthalmoscopic, electroretinography, histological, cell death and microbiological parameters. Expression of cytokines/chemokines and cellular infiltration was assessed. Intravitreal injection of chimeric Ply187AN-KSH3b (both at 6 and 12 h post infection) significantly improved the outcome of staphylococcal endophthalmitis, preserved retinal structural integrity, and maintained visual function. Phage lysin treatment significantly reduced the bacterial burden and the levels of inflammatory cytokines and neutrophil infiltration in the eyes. This is the first study demonstrating the therapeutic use of phage-based antimicrobials in ocular infections

### BACKGROUND OF THE INVENTION

1. Field of the Invention

This invention relates to the field of bacterial endophthalmitis and MDR Staphylococcus aureus including methicillin resistant S. aureus (MRSA), the leading cause of severe endophthalmitis, and the method of treating endophthalmitis with an antimicrobial fusion protein, the chimeric phage endolysin, Ply187AN-KSH3b, comprising a truncated Ply187 endolysin from the S. aureus prophage 187, where Ply187AN-KSH3b is effective for protecting individuals from development of endophthalmitis and for preventing vision loss.

2. Description of the Relevant Art

Bacterial endophthalmitis is a vision-threatening complication of ocular trauma and surgeries, particularly cataract surgery, a common surgical procedure performed on the aging population worldwide (Chiquet et al. 2008. Invest. Ophthalmol. Vis. Sci. 49:1971-1978). Moreover, the increased use of intravitreal injections (Campbell et al. 2010. Arch. Ophthalmol. 128:359-362) for the management of retinal diseases has also been implicated as a contributing factor to the increased incidence of endophthalmitis (Sadaka et al. 2012. Prog. Retin. Eye Res. 31:316-331). Although relatively uncommon, post intravitreal injection endophthalmitis can cause significant ocular morbidity (Sadaka et al., supra; Simunovic et al. 2012. Br. J. Ophthalmol. 96:862-866). The reported incidence of endophthalmitis per eye in multicentre clinical trials with anti-VEGF (Vascular Endothelial Growth Factor) therapy ranged from 0.7% to 1.6% (Kumar et al. 2010. J. Infect. Dis. 201:255-263; Scott and Flynn. 2007. Retina 27:10-12). As most postoperative endophthalmitis is caused by the bacteria from the ocular surface (Speaker et al. 1991. Ophthalmology 98:639-649; Callegan et al. 2007. Prog. Retin. Eye Res. 26:189-203), prophylactic measures include the use of topical antibiotics to reduce the density of the ocular flora (Kumar et al., supra; Lloyd and Braga-Mele. 2009. Can. J. Ophthalmol. 44:288-292; Yin et al. 2013. JAMA Ophthalmol. 131:456-461). However, recent studies have demonstrated that the use of topical antibiotics, specifically after intravitreal injection, does not reduce the risk of endophthalmitis, but rather, that there is a trend toward a higher incidence of endophthalmitis (Storey et al. 2014. Ophthalmology 121:283-289). This could be in part due to the fact that the repeated use of topical antibiotics increases the presence of antibiotic-resistant bacterial strains on the ocular surface (Alabiad et al. 2011. Am. J. Ophthalmol. 152:999-1004).

In the recent past, fluoroquinolones, such as moxifloxacin, were reported to be effective in preventing S. aureus endophthalmitis (Kowalski et al. 2008. Jpn. J. Ophthalmol. 52:211-216). Although this class of antibiotics covers a broad spectrum of organisms, they are largely ineffective against MRSA or MDR strains of S. aureus, the leading cause of severe endophthalmitis (Kumar et al., supra; Deramo et al. 2008. Am. J. Ophthalmol. 145:413-417; DeLeo and Chambers. 2009. J. Clin. Invest. 119:2464-2474). Furthermore, there is increasing evidence to suggest that ocular surface microflora are becoming more resistant to fourth-generation fluoroquinolones, with up to 30% of cultured ocular isolates being resistant (Yin et al., supra; Alabiad et al., supra; Moss et al. 2009. Ophthalmology 116:1498-1501). These findings support the necessity to search for new alternative prophylactic/therapeutic modalities against resistant bacteria in general, and S. aureus in particular, to prevent postoperative endophthalmitis.

Bacteriophage (phage) are viruses that infect bacteria. The phage endolysin is a peptidoglycan hydrolase that is produced near the end of the phage lytic cycle within the bacteria, in order to degrade the cell wall and allow the nascent phage particles to escape the lysed bacteria and infect new bacterial host cells. The endolysins are usually highly specific to the host bacteria (genus) and have evolved to bind to unique and essential bacterial cell wall targets. In recent years phage endolysins have attracted considerable interest as novel antibacterial agents (Schmelcher et al. 2012. Future Microbiol. 7:1147-1171), and have been used to treat a variety of bacterial infections, as demonstrated by an increasing number of experimental animal studies (Doehn et al. 2013. J. Antimicrob. Chemother. 68:2111-2117; Loessner, M. J. 2005. Curr. Opin. Microbiol. 8:480-487; Jun et al. 2011. Antimicrob. Agents Chemother. 55:1764-1767; Schuch et al. 2013. PLoS One 8:e60754; Nelson et al. 2001. Proc. Natl. Acad. Sci. USA 98:4107-4112; Oechslin et al. 2013. Antimicrob. Agents Chemother: 57(12):6276-6283; Pastagia et al. 2011. Antimicrob. Agents Chemother. 55:738-744; Gilmer et al. 2013. Antimicrob. Agents Chemother. 57:2743-2750; Vouillamoz et al. 2013. Int. J. Antimicrob. Agents 42:416-421; Jado et al. 2003. J. Antimicrob. Chemother. 52:967-973; Gervasi et al. 2014. Appl. Microbiol. Biotechnol. 98:2495-2505; Donovan, D. M. 2007. Recent Pat. Biotechnol. 1:113-122). Similarly, human studies (Gorski et al. 2009. Ophthalmologica 223:162-165) have documented the use of anti-staphylococcal phages for the treatment of conjunctivitis and blepharitis in 28 patients with no side effects (Proskurov, V. A. 1970. Vestn. Oftalmol. 6:82-83).

Among the bacterial pathogens, S. aureus is the leading cause of postoperative and post-traumatic endophthalmitis, a condition which often leads to vision loss (Sadaka et al., supra). The visual prognosis following bacterial endophthalmitis greatly depends on early detection and initiation of intravitreal antibiotic regimens (Endophthalmitis Vitrectomy Study Group. 1996. Am. J. Ophthalmol. 122:830-846). However, the increased incidence of ocular infections caused by antibiotic-resistant staphylococci, such as MRSA, highlights the need to develop alternative therapeutics, such as the utilization of bacteriophage or phage-encoded lytic enzymes (Doehn et al. 2013. J. Antimicrob. Chemother. 68:2111-2117; Pastagia et al., supra; Fischetti et al. 2006. Nat. Biotechnol. 24:1508-1511; Schmelcher et al., supra).

### SUMMARY OF THE INVENTION

We have discovered a method of treating Staphylococcus aureus-associated eye disease with the chimeric phage endolysin, Ply187AN-KSH3b, comprising the complete truncated Ply187AN peptidoglycan hydrolase polypeptide consisting of the cysteine, histidine-dependent amidohydrolases/peptidase (CHAP) endopeptidase domain of endolysin Ply187 together with the SH3b cell wall binding domain of native LysK, where the fusion polypeptide Ply187AN-KSH3b is capable of lysing and killing the S. aureus and is effective for treating and ameliorating symptoms of eye disease, protecting individuals from development of endophthalmitis and preventing vision loss.

In accordance with this discovery, it is an object of the invention to provide a composition useful for the treatment of disease caused by the Staphylococcus strains and MDR staphylococcal strains MRSA, for which the fusion protein Ply187AN-KSH3b is specific and effective, and to administer said Ply187AN-KSh3b to treat S. aureus-associated eye disease, including endophthalmitis caused by a MDR S. aureus.

It is an object of the invention to provide a method for protecting an individual from developing eye infection or eye disease caused by staphylococcal strains, including the MDR S. aureus, comprising: administering prophylactically to said individual, prior to surgery or intravitreal injections, an effective amount of a composition comprising the chimeric phage endolysin Ply187AN-KSH3b, wherein said administration is effective to protect said individual from developing eye infection or eye disease.

It is also an object of the invention to provide a method of ameliorating symptoms of eye infection or eye disease wherein the symptoms are an increased ocular bacterial burden, an infiltration of neutrophils, an increase in inflammatory cytokines and chemokines, impaired retinal function as measured by an ERG, ocular morbidity, retinal folding, corneal opacity, hyopyon, and scarring of the eye

Also part of this invention is a kit, comprising a composition comprising Ply187AN-KSH3b for the treatment of S. aureus-associated endophthalmitis, including endophthalmitis caused by a S. aureus, including MDR strains and MRSA, treatment for which Ply187AN-KSH3b is specific and effective.

Other objects and advantages of this invention will become readily apparent from the ensuing description.

### BRIEF DESCRIPTION OF THE DRAWINGS

The patent or application file contains at least one drawing executed in color. Copies of this patent or patent application publication with color drawing(s) will be provided by the U.S. Patent and Trademark Office upon request and payment of the necessary fee.

FIGS. 1A-1E depict the antimicrobial activity of the chimeric Ply187AN-KSH3b towards (FIG. 1A) Methicillin sensitive S. aureus (MSSA) and (FIGS. 1B-1E) MRSA/MDR strains/isolates. FIG. 1A-E shows results of the turbidity reduction assay performed by incubating 100 μl of bacteria (OD₆₀₀˜0.4) in assay buffer (400 mM NaCl, 20 mM Tris HCl, 1% glycerol, pH 7.5) with 100 μl of either PBS containing 30% glycerol (EB) as the control or chimeric Ply187AN-KSH3b (at the Minimum Inhibitory Concentration, MIC, per Table 1) in EB and monitoring the reduction in the turbidity every 5 min up to 1 h. Error bars represent SEM for three independent experiments performed in triplicates. FIG. 1F shows resistance development assay performed against S. aureus strain RN6390 by repeated exposure of bacteria (10⁵ CFU/well) to 2 fold serial dilution of Ply187AN-KSH3b, gentamicin (Gen), and lysostaphin (Lyso). Cells surviving at half (½) of the MIC value were used as an inoculum for each subsequent round of exposure. The change in susceptibility is recorded and presented as fold change resistance from day 1 to 10 of exposure and also after 5 days of growth (without any selection pressure) following the 10^{th} day (represented as 10+5). The data represent means±SEM of two independent experiments performed in triplicate.

FIGS. 2A-2E depicts the biofilm disruption activity of chimeric Ply187AN-KSH3b. In FIG. 2A-2E, either chimeric Ply187AN-KSH3b or EB (EB C) or Gentamicin (for MDR/MRSA strains/isolates) was applied to cover slips containing S. aureus RN6390 (FIG. 2A), USA 300 (FIG. 2B), R 2932 (FIG. 2C), R 2952 (FIG. 2D) and R3000 (FIG. 2E) biofilms for 30 min. Bacterial killing was assessed via Live/Dead™ staining and subsequent fluorescence microscopy. FIG. 2F depicts the viability of biofilm associated bacteria enumerated by CFU count. Error bars represent the mean±SEM of two independent experiments performed in triplicate.

FIGS. 3A-3E show the effects of intravitreal injection of chimeric Ply187AN-KSH3b on the severity of S. aureus endophthalmitis. The eyes (n=10) of C57BL/6 mice were infected with S. aureus strain RN6390 and following either 6 h or 12 h post-infection, Ply187AN-KSH3b or EB (EB C) was administered by intravitreal injection. FIG. 3A depicts individual eyes assigned clinical scores (range 0 to 4; black dots represent an eye) and presented as mean clinical scores (bar with SEM). FIG. 3B shows slit lamp microscopy examination performed and micrographs taken from representative eyes. FIG. 3C shows the histological analysis of chimeric Ply187AN-KSH3b treatment following H&E staining. FIG. 3D shows the TUNEL staining following infection and the chimeric Ply187AN-KSH3b treatment. FIG. 3E depicts the total number of dead cells in the retinal sections that were counted and are presented as mean±SD of the TUNEL positive cells. A student's t test (**, p<0.005) was used for the statistical analysis and comparisons were made between the S. aureus (SA) control, SA+EBC, and SA+Ply187AN-KSH3b-treated group.

FIGS. 4 A, B and C depict the effect of chimeric Ply187AN-KSH3b on bacterial growth in vivo and in the vitreous humor (VH) in vitro and with increasing inoculums in vitro respectively. FIG. 4A shows that eyes inoculated with S. aureus (strain RN6390) were treated at 6 h and 12 h post infection with chimeric Ply187AN-KSH3b or EB (EB C). Twenty-four hours after the treatment, the eyes were enucleated, homogenized and the bacterial burden was estimated via serial dilution plating. FIG. 4B depicts a test showing the effect of the vitreous humor on Ply187AN-KSH3b antimicrobial activity where Ply187AN-KSH3b was mixed with calf vitreous humor and inoculated with 10⁵ CFU of S. aureus. The survival of the bacteria was enumerated by CFU count at 2, 4 and 6 h of incubation. Statistical analysis was performed using unpaired student's t test (*, p<0.05; **, p<0.005; ***, p<0.0005). FIG. 4C shows the inoculum effect on Ply187AN-KSH3b activity. An increasing number of dilutions (1:10,000 to 1:10) of logarithmic phase of S. aureus (RN6390 strain) were treated with Ply187AN-KSH3b for 1 h followed by CFU enumeration on TSA plates. Statistical analysis was performed using unpaired student's t test (*, p<0.05; **, p<0.005; ***, p<0.0005).

FIG. 5 shows the effect of chimeric Ply187AN-KSH3b on inflammatory cytokines in vivo. Following treatment with chimeric Ply187AN-KSH3b or EB (EB C), the eyes (n=4 per time point) were enucleated and 10 μg protein lysate was used for the detection of indicated inflammatory cytokines/chemokines by ELISA. *p<0.05, **p<0.005 (student t-test, SA or EB vs. Ply187AN-KSH3b-treated)

FIG. 6 depicts infiltration of polymorphonuclear cells (PMNs) in the Ply187AN-KSH3b-treated mouse retina/vitreous cavity. Infected eyes were given intravitreal injections of either EB or chimeric Ply187AN-KSH3b for either 6 or 12 h post infection. Twenty four hour post Ply187AN-KSH3b treatment, the eyes (n=6) were enucleated and the retina/vitreous humor from two eyes were pooled to make single-cell suspensions. The cells were stained with anti-CD45 and anti-Ly6G mAbs. Post-acquisition, the cells were size-gated to differentiate them from debris. The percentage of dually positive PMNs was determined using a CD45-versus-Ly6G dot plot (upper-right quadrant). The data are summarized in a bar graph representative of duplicate experiments. *p<0.05 (student t-test)

FIG. 7 shows the effect of Ply187AN-KSH3b treatment on retinal function. ERG responses to a 6-dB flash were recorded from uninfected control eyes (FIG. 7A), from S. aureus (SA)-infected eyes (FIG. 7B) and from S. aureus (SA)-infected, Ply187AN-KSH3b-treated eyes 24 h post treatment (FIG. 7C) in both the infected and Ply187AN-KSH3b-treated mice (n+4-6). The percentage amplitude of the a- and b-waves retained in S. aureus (SA)-infected, treated and untreated mice were compared to normal uninfected mice and presented as mean±SD (FIG. 7D). The level of significance between SA-infected control and lysin-treated mice was determined by unpaired student's t test (**, p<0.005).

### DETAILED DESCRIPTION OF THE INVENTION

Our data demonstrate that a single intravitreal injection of a previously described chimeric endolysin Ply187AN-KSH3b (SEQ ID NO:1) (Mao et al. 2013. FEMS MicrobioL Lett. 342:30-36) protects mice from the development of endophthalmitis, markedly diminishes the progression of endophthalmitis in mice, and prevents vision loss. In view of the limited supply of new antimicrobial agents and the increasing antibiotic resistance among ocular pathogens, our study demonstrates, for the first time, the potential use of phage endolysin therapy in bacterial endophthalmitis.

In the last two decades, phage endolysins have emerged as unique antimicrobial agents that possess exceptionally high specificity and an ability to act against MDR microbes (Doehn et al., supra; Pastagia et al., supra; Fischetti et al., supra; Schmelcher et al., supra). Lysins consist of a catalytic domain and a binding domain; the catalytic domain cleaves specific bonds in bacterial peptidoglycan and is often conserved among the same class of hydrolases. Similarly, the cell wall-binding domain (CBD) is often conserved allowing each lysin to target a specific substrate in the bacterial cell wall and confers some species—specificity to these lysin molecules. The potent chimeric endolysin, Ply187AN-KSH3b was generated by fusing the cysteine, histidine-dependent amidohydrolases/peptidase (CHAP) endopeptidase domain of endolysin Ply187 from staphylococcal phage 187 with the SH3b CBD of LysK from staphylococcal phage K (Mao et al., supra). Ply187AN-KSH3b was found to be a more effective antimicrobial than the full-length Ply187 or the truncated Ply187 (Ply187AN); and also outperforms the known high activity lysin, LysK (Mao et al., supra). Here, we show that chimeric Ply187AN-KSH3b exerts strong lytic activity against RN6390, MRSA USA 300, MDR R2932, R2952, R2300 strain/isolates of S. aureus. Our resistance development frequency data showed that S. aureus was unable to develop resistance against Ply187AN-KSH3b following repeated exposure. Moreover, chimeric Ply187AN-KSH3b disrupts biofilm formation by these strains/isolates. Because biofilm formation plays an important role in the pathogenesis of ocular infections including endophthalmitis (Leid et al. 2002. DNA Cell Biol. 21:405-413; Behlau and Gilmore. 2008. Arch. Ophthalmol. 126:1572-1581; Suzuki et al. 2005. J. Cataract Refrac. Surg. 31:2019-2020), the dispersion of biofilms by chimeric Ply187AN-KSH3b indicates its therapeutic potential in the treatment of endophthalmitis.

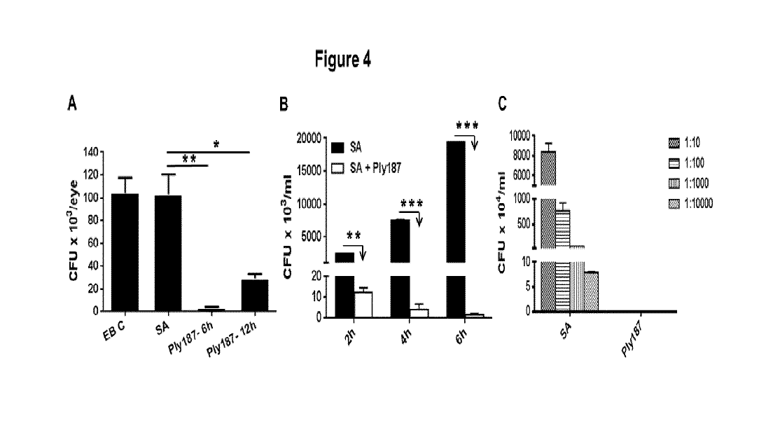

Since pathogenesis of ocular bacteria results in the release of toxins and degradative enzymes that can damage the integrity of ocular tissues and cause irreversible damage (Bertino, J. S. Jr. 2009. Clin. Ophthalmo. 3:507-521), it is important to choose antimicrobials with rapid bactericidal activity. In cases of suspected bacterial endophthalmitis, intravitreal injection of both vancomycin and an aminoglycoside or a third-generation cephalosporin is recommended, while vitrectomy may be needed for severe cases (Callegan et al., supra). However, MDR ocular S. aureus strains are becoming increasingly more prevalent (McDonald and Blondeau. 2010. J. Cataract Refrac. Surg. 36:1588-1598) and chimeric Ply187AN-KSH3b is effective against MDR strains of S. aureus. We show that intravitreal injection of chimeric Ply187AN-KSH3b significantly reduces the bacterial burden in the eyes of C57BL/6 mice at either 6 or 12 hours post-infection. Moreover, the bacterial burden in the 12 h treatment group was slightly higher than the 6 h treatment group. To test whether this phenomena was due to reduced activity of the chimeric Ply187AN-KSH3b in an environment where bacteria are proliferating, we performed an inoculum effect study. To this end our data (FIG. 4C) showed that the activity of chimeric Ply187AN-KSH is independent of bacterial growth. One possible explanation could be that by 12 h of infection some bacteria might not have access to endolysin due to having been internalized/phagocytized by infiltrating PMNs and monocytes as reported earlier (Rogers and Tompsett. 1952. J. Exp. Med. 95:209-230; Melly et al. 1960. J. Exp. Med. 112:1121-1130; Kapral and Shayegani. 1959. J. Exp. Med. 110:123-138; Gresham et al. 2000. J. Immunol. 164:3713-3722). As incubation of chimeric Ply187AN-KSH with vitreous humor did not affect its bactericidal activity, the rapid efficacy of chimeric Ply187AN-KSH in killing bacteria inside the eye suggests that there is no specific cellular factor (e.g., proteases) that can prevent the chimeric endolysin activity. Hence, we demonstrate that endolysins, when delivered intravitreally, are efficacious in limiting intraocular bacterial growth.

S. aureus produces a variety of virulence factors that are either cell wall-associated molecules or secreted bacterial proteins (often toxins). The coordinated actions of these virulence factors lead to tissue destruction and the clinical manifestations of endophthalmitis (Shamsuddin and Kumar. 2011. J. Immunol, 186:7089-7097). Our histological analysis revealed that eyes treated with chimeric Ply187AN-KSH3b had reduced retinal damage compared to untreated eyes. The rapid decline in ERG response in the control group suggests the dysfunction of retinal cells. This in part could be due to the death of retinal cells as reported in previous studies (Talrega et al. 2014. Invest. Ophthalmol. Vis. Sci. In press; Whiston et al. 2008. Infect. Immun. 76:1781-1790). Our TUNEL data also support these findings and suggests that in bacterial endophthalmitis, the cells undergoing apoptosis are mainly retinal cells. Moreover, as the chimeric Ply187AN-KSH3b-treated eyes retained significant retinal function, this could also be due to the reduced retinal cell death in the treatment group. Similar to reduced tissue damage, the levels of inflammatory cytokines/chemokines (IL-6, IL1β, TNFα, MIP-2, and KC) were also attenuated by chimeric Ply187AN-KSH3b treatment. This is advantageous because an excessive inflammatory response can be harmful to retinal neurons. Neutrophils (PMNs) play an important role in bacterial clearance but, paradoxically, they are also involved in the pathology of endophthalmitis (Sadaka et al., supra; Callegan et al., supra). Our data show that chimeric Ply187AN-KSH3b treatment reduced the PMN infiltration by 40-50% as compared to control mice. The decline in PMN response could be due to reduced bacterial burden. Moreover, we observed that chimeric Ply187AN-KSH3b-treated animals were able to retain ˜90-95% of both a- and b-wave amplitudes indicating a protective role of phage endolysin treatment in the preservation of retinal function.

If one were to develop phage endolysins as a possible therapeutic agent in humans, safety must always be of great concern. Studies have shown that a rapid release of bacterial ghosts and the sudden release of intracellular bacterial components may amplify the inflammatory response causing septic shock and multiple organ failure (Fischetti, V. A. 2010. Int. J. Med. Microbiol. 300:357-362; Entenza et al. 2005. Antimicrob. Agents Chemother. 49:4789-4792; Nau and Eiffert. 2002. Clin. Microbiol. Rev. 15:95-110). However, none of the endolysin studies performed in vivo so far have reported such side effects. Our cytotoxicity analysis showed that chimeric Ply187AN-KSH3b does not cause retinal cell death both in vitro (cultured microglia, Müller glia and RPE) and in vivo (data not shown). Similarly, the intravitreal injection of chimeric Ply187AN-KSH3b alone does not evoke an inflammatory response in the eye, suggesting that there are no adverse effects of endolysins in the eye.

In conclusion, we demonstrate that a single intravitreal injection of phage endolysin was efficient in protecting the mouse eyes from staphylococcal endophthalmitis. Thus, based on this first proof of principle study, we show that phage lytic enzyme-based therapy can be used for the treatment of endophthalmitis in patients with antibiotic resistant bacterial infections.

As used herein, “recombinant” refers to a nucleic acid molecule which has been obtained by manipulation of genetic material using restriction enzymes, ligases, and similar genetic engineering techniques as described by, for example, Sambrook et al. 1989. Molecular Cloning: A Laboratory Manual, Second Edition, Cold Spring Harbor Laboratory Press, Cold Spring Harbor, N.Y. or DNA Cloning: A Practical Approach, Vol. I and II (Ed. D. N. Glover), IRL Press, Oxford, 1985. “Recombinant,” as used herein, does not refer to naturally occurring genetic recombinations.

As used herein, the term “chimeric” refers to two or more DNA molecules which are derived from different sources, strains, or species, which do not recombine under natural conditions, or to two or more DNA molecules from the same species, which are linked in a manner that does not occur in the native genome.

As used herein, the terms “encoding”, “coding”, or “encoded” when used in the context of a specified nucleic acid mean that the nucleic acid comprises the requisite information to guide translation of the nucleotide sequence into a specified protein. The information by which a protein is encoded is specified by the use of codons. A nucleic acid encoding a protein may comprise non-translated sequences (e.g., introns) within translated regions of the nucleic acid or may lack such intervening non-translated sequences (e.g., as in cDNA).

A “protein” or “polypeptide” is a chain of amino acids arranged in a specific order determined by the coding sequence in a polynucleotide encoding the polypeptide. Each protein or polypeptide has a unique function.

The invention includes functional fragments of the Ply187AN-KSh3b fusion peptidoglycan hydrolase polypeptide and functional fusion polypeptides encompassing a functional Ply187AN-KSh3b fusion peptidoglycan hydrolase and functional fragments thereof, as well as mutants and variants having the same biological function or activity. As used herein, the terms “functional fragment”, “mutant” and “variant” refers to a polypeptide which possesses biological function or activity identified through a defined functional assay and associated with a particular biologic, morphologic, or phenotypic alteration in the cell. The term “functional fragments of Ply187AN-KSh3b fusion peptidoglycan hydrolase” refers to all fragments of Ply187AN-KSh3b fusion peptidoglycan hydrolase that retain Ply187AN-KSh3b fusion peptidoglycan hydrolase activity and function to lyse staphylococci.

Modifications of the Ply187AN-KSh3b fusion peptidoglycan hydrolase primary amino acid sequence may result in further mutant or variant proteins having substantially equivalent activity to the Ply187AN-KSh3b fusion peptidoglycan hydrolase polypeptides described herein. Such modifications may be deliberate, as by site-directed mutagenesis, or may occur by spontaneous changes in amino acid sequences where these changes produce modified polypeptides having substantially equivalent activity to the Ply187AN-KSh3b fusion peptidoglycan hydrolase polypeptide. Any polypeptides produced by minor modifications of the Ply187AN-KSh3b fusion peptidoglycan hydrolase primary amino acid sequence are included herein as long as the biological activity of Ply187AN-KSh3b fusion peptidoglycan hydrolase is present; e.g., having a role in pathways leading to lysis of staphylococci.

As used herein, “sequence identity” or “identity” in the context of two nucleic acid or polypeptide sequences makes reference to the residues in the two sequences that are the same when aligned for maximum correspondence over a specified comparison window. When percentage of sequence identity is used in reference to proteins, it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule.

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

Substantial identity of amino acid sequences for these purposes normally means sequence identity of at least 80%, preferably at least 85%, more preferably at least 90%, and most preferably at least 95%. Preferably, optimal alignment is conducted using the homology alignment algorithm of Needleman et al. (1970. J. Mol. Biol. 48:443).

A “substantial portion” of an amino acid or nucleotide sequence comprises an amino acid or a nucleotide sequence that is sufficient to afford putative identification of the protein or gene that the amino acid or nucleotide sequence comprises. Amino acid and nucleotide sequences can be evaluated either manually by one skilled in the art, or by using computer-based sequence comparison and identification tools that employ algorithms such as BLAST. In general, a sequence of ten or more contiguous amino acids or thirty or more contiguous nucleotides is necessary in order to putatively identify a polypeptide or nucleic acid sequence as homologous to a known protein or gene. Moreover, with respect to nucleotide sequences, gene-specific oligonucleotide probes comprising 30 or more contiguous nucleotides may be used in sequence-dependent methods of gene identification and isolation. In addition, short oligonucleotides of 12 or more nucleotides may be use as amplification primers in PCR in order to obtain a particular nucleic acid fragment comprising the primers. Accordingly, a “substantial portion” of a nucleotide sequence comprises a nucleotide sequence that will afford specific identification and/or isolation of a nucleic acid fragment comprising the sequence. The instant specification teaches amino acid and nucleotide sequences encoding polypeptides that comprise a particular phage protein. The skilled artisan, having the benefit of the sequences as reported herein, may now use all or a substantial portion of the disclosed sequences for purposes known to those skilled in this art. Thus, such a portion represents a “substantial portion” and can be used to establish “substantial identity”, i.e., sequence identity of at least 80%, compared to the reference sequence. Accordingly, the instant invention comprises the complete sequences as reported in the accompanying Sequence Listing, as well as substantial portions at those sequences as defined above.

Fragments and variants of the disclosed nucleotide sequences and proteins encoded thereby are also encompassed by the present invention. By “fragment” a portion of the nucleotide sequence or a portion of the amino acid sequence and hence protein encoded thereby is intended. Fragments of a nucleotide sequence may encode protein fragments that retain the biological activity of the native protein and hence have Ply187AN-KSh3b fusion peptidoglycan hydrolase-like activity. Alternatively, fragments of a nucleotide sequence that are useful as hybridization probes may not encode fragment proteins retaining biological activity.

By “variant protein” a protein derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein is intended. Variant proteins encompassed by the present invention are biologically active, that is they possess the desired biological activity, that is, Ply187AN-KSh3b fusion peptidoglycan hydrolase activity as described herein. Such variants may result from, for example, genetic polymorphism or from human manipulation. Biologically active variants of the Ply187AN-KSh3b fusion peptidoglycan hydrolase protein of the invention will have at least about 90%, preferably at least about 95%, and more preferably at least about 98% sequence identity to the amino acid sequence for the native protein as determined by sequence alignment programs described elsewhere herein. A biologically active variant of a protein of the invention may differ from that protein by as few as 1-15 amino acid residues, or even 1 amino acid residue.

The polypeptides of the invention may be altered in various ways including amino acid substitutions, deletions, truncations, and insertions. Novel proteins having properties of interest may be created by combining elements and fragments of proteins of the present invention, as well as with other proteins. Methods for such manipulations are generally known in the art. Thus, the genes and nucleotide sequences of the invention include both the naturally occurring sequences as well as mutant forms. Likewise, the proteins of the invention encompass naturally occurring proteins as well as variations and modified forms thereof. Such variants will continue to possess the desired Ply187AN-KSh3b fusion peptidoglycan hydrolase activity. Obviously, the mutations that will be made in the DNA encoding the variant must not place the sequence out of reading frame and preferably will not create complementary regions that could produce secondary mRNA structure.

The deletions, insertions, and substitutions of the protein sequences encompassed herein are not expected to produce radical changes in the characteristics of the protein. However, when it is difficult to predict the exact effect of the substitution, deletion, or insertion in advance of doing so, one skilled in the art will appreciate that the effect will be evaluated by routine screening assays where the effects of Ply187AN-KSh3b fusion peptidoglycan hydrolase protein can be observed.

The staphylococcal control compositions of the invention comprise the antimicrobial composition of the invention dissolved or suspended in an aqueous carrier or medium. The composition may further generally comprise an acidulant or admixture, a rheology modifier or admixture, a film-forming agent or admixture, a buffer system, a hydrotrope or admixture, an emollient or admixture, a surfactant or surfactant admixture, a chromophore or colorant, and optional adjuvants. The preferred compositions of this invention comprise ingredients which are generally regarded as safe, and are not of themselves or in admixture incompatible with human and veterinary applications. Likewise, ingredients may be selected for any given composition which are cooperative in their combined effects whether incorporated for antimicrobial efficacy, physical integrity of the formulation or to facilitate healing and health in medical and veterinary applications, including for example in the case of endophthalmitis, healing and health of the eye or other human or animal body part. Generally, the composition comprises a carrier which functions to dilute the active ingredients and facilitates stability and application to the intended surface. The carrier is generally an aqueous medium such as water, or an organic liquid such as an oil, a surfactant, an alcohol, an ester, an ether, or an organic or aqueous mixture of any of these. Water is preferred as a carrier or diluent in compositions of this invention because of its universal availability and unquestionable economic advantages over other liquid diluents.

A therapeutically effective dose refers to that amount of active ingredient, which ameliorates the symptoms or condition. Therapeutic efficacy and toxicity, e.g. ED₅₀ and LD₅₀, may be determined by standard pharmacological procedures in cell cultures or experimental animals. The dose ratio between therapeutic and toxic effects is the therapeutic index and may be expressed by the ratio LD₅₀/ED₅₀. Pharmaceutical compositions exhibiting large therapeutic indexes are preferred.

Using highly specific antimicrobials which target specific sites of the specific organisms involved rather than relying on the generalized use of broad range antimicrobials can enhance our effectiveness in treating disease and also enable us to reduce the occurrence of antibiotic resistance.

### EXAMPLES

Having now generally described this invention, the same will be better understood by reference to certain specific examples, which are included herein only to further illustrate the invention and are not intended to limit the scope of the invention as defined by the claims.

### Example 1

### Bacterial Strain; Plasmid Constructs and DNA Manipulation

For in vivo studies, an antibiotic sensitive S. aureus (SA) strain RN6390 was used to induce endophthalmitis; whereas, the in vitro studies were performed using antibiotic-resistant strains (Table 1), including USA300, a community-associated methicillin resistant S. aureus (CA-MRSA), and three clinical isolates R2932 (CA-MRSA), R2952 and R3000 (both being hospital-associated MRSA, HA-MRSA), HA-MRSA) kindly provided by Dr. Michael J. Rybak (Department of Pharmaceutical Sciences, Wayne State University, Detroit, Mich.). All bacteria were routinely cultured in TSB broth (Tryptic Soy broth, Sigma, St Louis, Mo.) or on TSB agar plates.

TABLE 1
Antibacterial activity of chimeric Ply187AN-KSH3b against
S. aureus strains.

| Isolate ID | Phenotype | MIC (μg/ml) |
|---|---|---|
| RN6390 | MSSA | 4.16 ± 1.04 |
| USA 300 | CA-MRSA | 4.16 ± 1.04 |
| R 2932 | CA-MRSA | 4.16 ± 1.04 |
| R 2952 | HA-MRSA | 8.33 ± 2.08 |
| R 3000 | HA-MRSA | 4.16 ± 1.04 |

Data are shown as mean values from three experiments ± SEM.

The candidate therapeutic Ply187AN-KSH3b (referred to as chimeric Ply187AN-KSH3b) is a staphylococcal peptidoglycan hydrolase fusion protein containing the endopeptidase domain from a staphylococcal prophage endolysin, Ply187, and the SH3b cell wall-binding domain of the staphylococcal phage K endolysin, LysK. To enhance the heterologous expression of Ply187 endolysin in E. coli, the sequences encoding the truncated Ply187 N-terminal domain (Ply187AN; 1-157aa) were converted to an E. coli codon bias, commercially synthesized, and subcloned into pUC57 with engineered 5′ NdeI (CATATG; ATG=start of translation) and 3′ XhoI (CTCGAG; codes for aa's LE) restriction enzyme sites (Genscript; Piscataway, N.J.). Subcloning of the Ply187AN construct into the pET21a expression vector was via conventional means for protein expression. Similarly, the Ply187AN was fused to the LysK SH3b by subcloning the Ply187AN NdeI-XhoI DNA fragment harboring all CHAP lytic domain coding sequences into a similarly digested pre-constructed pET21a-KSH3b vector described previously (Becker et al. 2009a. FEMS Microbiol. Lett. 294:52-60; Mao et al., supra).

### Example 2

### Protein Purification and Analysis

Protein induction, purification and storage followed the protocols as described previously (Becker et al. 2009b, supra). Briefly, Escherichia coli cultures harboring vectors were harvested, then sonicated for 5 min using an automatic pulsing sonication (Bronson Sonifier; Bronson Sonic Power Co., Danbury, Conn., USA). His-tagged proteins were isolated using Ni-NTA nickel column chromatography (Qiagen). Wash and elution profiles were empirically determined to be 10 ml of 10 mM imidazole, 20 ml of 20 mM imidazole and elution with 1.2 ml of 250 mM imidazole in phosphate buffered saline (50 mM NaH₂PO₄, 300 mM NaCl, pH 8.0) with 1% glycerol to prevent precipitation of the purified protein. All samples were then desalted with Zeba desalting column (Pierce, Rockford, Ill.), equilibrated in 2×PBS buffer and filter sterilized. The sterilized protein preparation was stored at 4° C. in 2×PBS buffer 30% glycerol until the time of assay.

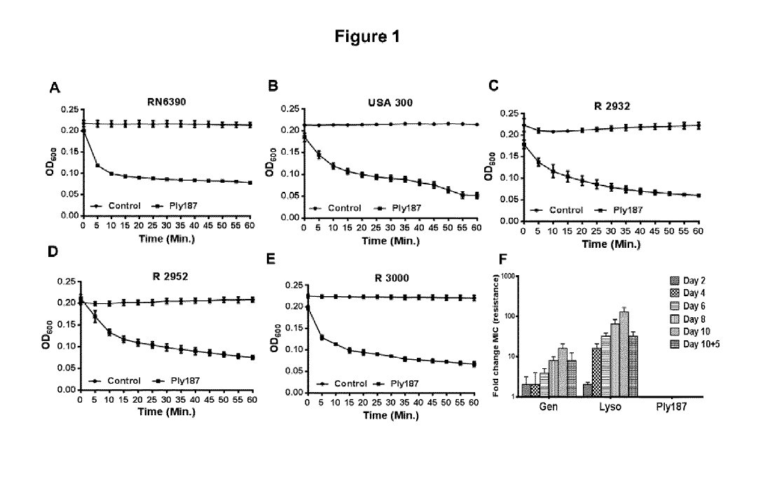

Nickel chromatography-purified proteins were analyzed using 15% SDS-PAGE and Kaleidoscope protein standards (Bio-Rad, Hercules, Calif.) (FIG. 1B), with or without 300 ml culture equivalents of mid log phase S. aureus cells (OD₆₀₀ₙₘ=0.4-0.6) embedded in the gel as described previously (Becker et al. 2009b, supra), to verify the absence of co-purifying lytic contaminants. Coomassie-stained SDS-PAGE of purified protein C-His-Ply187AN-KSH3b indicated that the construct was able to be expressed in E. coli and purified at greater than 95% purity. Zones of lysis on the zymogram gel run in parallel with the SDS-PAGE indicate that the predicted is the only protein with staphylolytic activity. For intravitreal injection, the purified chimeric Ply187AN-KSH3b protein was dialyzed gradually against PBS containing 30% glycerol (EB), and filter sterilized.

### Example 3

### Chimeric Ply187AN-KSH3b Exhibits Anti-Staphylococcal Activity; and S. aureus Fails to Develop Resistance Against Ply187AN-KSH3b In Vitro

To verify and quantify the lytic activity against live S. aureus, we have tested Ply187AN-KSH3b in the Turbidity Reduction Assay. The turbidity assay measures the drop in optical density (OD) resulting from lysis of the target bacteria with the phage endolysin-derived protein. Turbidity reduction assays by chimeric Ply187AN-KSH3b were performed in a 96 well plate as described previously by Becker et al. (2009a, 2009b, supra). Briefly, S. aureus strains/isolates RN6390, USA 300, and clinical isolates R2932, R2952 and R3000 were grown to logarithmic phase (OD₆₀₀ 0.4-0.6) at 37° C. in tryptic Soy broth (TSB). The culture was harvested by centrifugation and the pellet was resuspended in assay buffer (400 mM NaCl, 20 mM Tris HCl, 1% glycerol, pH 7.5). Bacterial cultures (100 μl/well) were mixed with 100 μl of (as per MIC) of chimeric Ply187AN-KSH3b protein diluted in the same assay buffer. The reduction in the turbidity was measured after every 5 min up to 1 h using a micro plate reader. EB diluted in the same assay buffer (without chimeric Ply187AN-KSH3b protein) was used as a control (EB C). Resistance development against chimeric Ply187AN-KSH3b was tested using repeated exposure in an MIC assay as described by Rodriguez-Rubio and co-workers (Rodriguez-Rubio et al., PLoS One 8:e64671.). In brief, bacterial cultures (10⁵ CFU/well) were exposed overnight to a 2-fold serial dilution of chimeric Ply187AN-KSH3b, lysostaphin, and gentamicin, where the latter serve as controls. In every round, 100 μl culture from the wells with growth (½ MIC value) were inoculated into fresh TSB and grown up to logarithmic phase. These cultures were used for the next round of MIC exposure. Bacteria surviving after 10 rounds were grown for an additional 5 rounds in TSB without any selection pressure to allow phenotype-reversion, then MIC were performed to measure the sensitivity to the chimeric Ply187AN-KSH3b after non-selective growth.

As shown in FIG. 1A-1E, incubation of S. aureus with chimeric Ply187AN-KSH3b resulted in a time-dependent reduction in the turbidity of suspension culture, indicating cell lysis and the lytic activity of Ply187AN-KSH3b. FIG. 1F shows that strain RN6390 failed to develop any resistance against Ply187AN-KSH3b in up to 10 rounds of exposure. In contrast, strain RN6390 exhibited 128 and 16 fold change in resistance towards lysostaphin and gentamicin respectively.

### Example 4

### Chimeric Ply187AN-KSH3b Disrupts S. aureus Biofilms on Abiotic Surfaces

Biofilm disruption by the chimeric Ply187AN-KSH3b was observed using Live/Dead BacLight™ staining which takes advantage of SYTO™ 9, a green-fluorescent nucleic acid stain and propidium iodide that fluoresces red. SYTO™ 9 labels a bacterial population with an intact membrane and fluoresces green. In contrast, propidium iodide penetrates bacteria with damaged membranes and fluoresces red. Briefly, static biofilms of S. aureus (strain RN6390, USA 300, R 2932, R 2952, R 3000) were grown on glass cover slips in 6 well tissue culture plates in fresh TSB media seeded with S. aureus. After incubation at 37° C. for 24 h, the biofilms were treated for an additional 30 min with chimeric Ply187AN-KSH3b (as per MIC) diluted in EB or with EB/gentamicin alone. The biofilms were then stained with the Live/Dead BacLight™ bacterial viability kit (Invitrogen, Carlsbad, Calif.), per manufacturer's instruction. The cover slips were washed three times with PBS to remove excess stain and cell debris, mounted with mounting oil on a glass slide, and examined with an Eclipse 90i fluorescence microscope (Nikon, Melville, N.Y.).

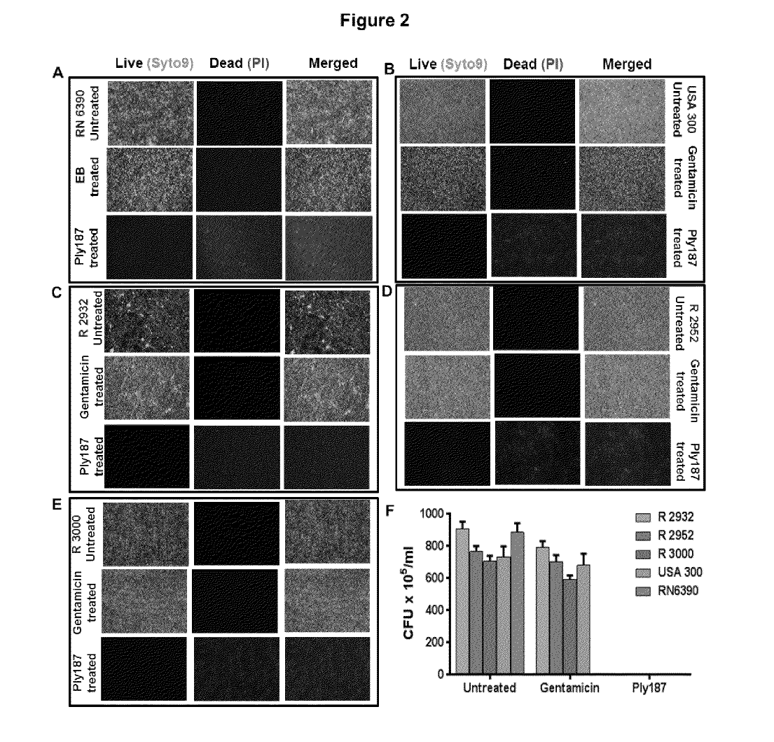

The capacity of chimeric Ply187AN-KSH3b endolysin to disrupt biofilms was determined by fluorescent imaging of Ply187AN-KSH3b-treated and untreated biofilms of S. aureus using Live/Dead BacLight™. FIGS. 2A-2E show 24 h biofilms of S. aureus treated for 30 min with Ply187AN-KSH3b (as per MIC) or EB or Gentamicin. The green fluorescence (due to SYTO™ 9) in EB or gentamicin-treated and untreated control biofilms (SA) contrasted with the chimeric Ply187AN-KSH3b-treated biofilms that had negligible green fluorescence and having red fluorescence only red (due to propidium iodide invasion of lysed/disrupted cells), indicates that Ply187AN-KSH3b successfully disrupted biofilms. Upon microscopic examination, the residual staining in the Ply187AN-KSH3b-treated biofilm was determined to be cellular debris, with little to no intact staphylococci. To provide further evidence of biofilm disruption and quantification, we enumerated the CFU counts from both treated and untreated biofilms. Our results indicate that, the untreated biofilms having very high CFU counts for all S. aureus strains tested in contrast to the Ply187 treated biofilms where the CFU counts were almost zero, indicating the complete disruption of biofilms (FIG. 2F).

### Example 5

### Chimeric Ply187AN-KSH3b-Treatment Attenuates Development of Endophthalmitis

The eyes of C57BL/6 mice (female, 8 week old) were intravitreally injected with 1 μl of PBS containing 5000 colony forming units (CFU) of S. aureus RN6390 to induce endophthalmitis as described previously (Kumar et al., supra). The chimeric Ply187AN-KSH3b (1 μg/μl) treatment in EB was given after 6 h (group I) or 12 h (group II) post infection. In the control group, the eyes were treated with EB (EB C). The eye infected with S. aureus without any treatment was used as a disease control. Clinical examinations were performed using slit lamp microscopy. The ocular disease was graded, and clinical scores from 0 to 4.0 were assigned using the previously described scale (Whiston et al., supra; Ramadan et al. 2006. Curr. Eye Res. 31:955-965). The clinical score of 4 is considered as 100 percent damage. Based on this, the percent damage and percent retention of eyes were calculated in both Ply187AN-KSH3b-treated vs untreated S. aureus infected eyes. Mice were treated in compliance with the ARVO Statement for the Use of Animals in Ophthalmic and Vision Research and all procedures were approved by the Institutional Animal Care and Use Committee (IACUC) of Wayne State University.

The embedding, sectioning, and Hematoxylin and Eosin (H & E) staining was performed by Excalibur Pathology Inc. (Oklahoma City, Okla.). For immunostaining, the eyes were fixed in Tissue-Tek® O.C.T. (Sakura, Torrance, Calif.) and five-micrometer-thick sagittal sections were collected from each eye and mounted onto microscope slides. TUNEL staining was performed on retinal cryo-sections using ApopTag® Fluorescein In situ Apoptosis Detection Kit as per manufacturer's instruction (Millipore, Billerica, Mass.).

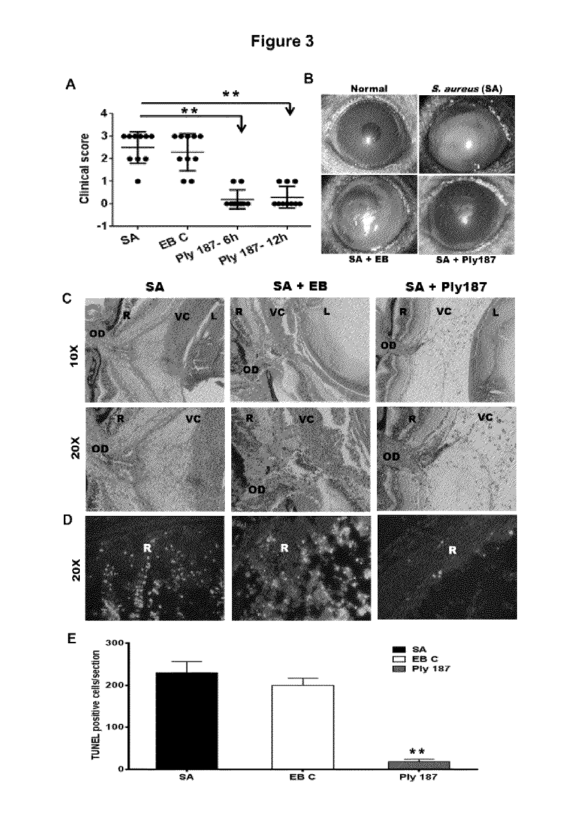

We hypothesized that similar to antibiotics, chimeric Ply187AN-KSH3b would have beneficial effects in protecting the eyes from endophthalmitis. To test this hypothesis, we used a C57BL/6 mouse model of S. aureus endophthalmitis. Chimeric Ply187AN-KSH3b was administrated intravitreally (1 μg/eye in 1 μl volume of EB) 6 h and 12 h after bacterial infection. Eyes without any treatment or eyes injected with EB served as controls. After 24 h, each infected eye was assigned a clinical score. The mean clinical scores of chimeric Ply187AN-KSH3b-treated eyes (FIG. 3A) was significantly (P<0.005) lower in both the 6 h (0.2) and 12 h treatment (0.3) groups when compared to the elution buffer control (EB C)-treated eyes (2.3) and infected control eyes (2.5). The conversion of clinical scores into the percentage of the eye destroyed or retained revealed 90-95% retention of eyes in the treatment group. FIG. 3B shows microscopic images of chimeric Ply187AN-KSH3b-treated eyes displaying clear/transparent corneas and/or anterior chambers, whereas the S. aureus-infected and EB-treated control eyes showed dense corneal opacity, hyopyon, and a severe inflammatory response.

Histological analysis and H&E staining in FIG. 3C revealed that the infected eyes of untreated or EB-treated mice showed a heavy cellular infiltrate in the posterior segment, as well as retinal folding. In contrast, treatment with chimeric Ply187AN-KSH3b preserved the integrity of the retina, and little infiltration was detected in the posterior segment. To further assess the extent of retinal damage, TUNEL staining was performed on retinal sections (FIG. 3D) which showed significantly reduced (average 17.66 TUNEL⁺ cells/eye) cell death in chimeric Ply187AN-KSH3b treatment vs untreated (average 230 TUNEL⁺ cells/eye) and EB-treated (average 200.33 TUNEL⁺ cells/eye) control groups (FIG. 3E). Taken together these results indicate that chimeric Ply187AN-KSH3b treatment significantly improves the disease outcome.

### Example 6

### Chimeric Ply187AN-KSH3b Reduces Bacterial Load

The bacterial burden in both chimeric Ply187AN-KSH3b- and vehicle-treated eyes was estimated using a bacterial plate count method. At the desired time point, the eyes were enucleated and homogenized in sterile PBS by stainless steel beads using Tissue lyser (Qiagen, Valencia, Calif.). The homogenate was serial diluted in sterile PBS and plated on TSA plates. The results were expressed as the mean number of CFU/eye ±SEM.

To determine the effect of Vitreous Humor (VH) on antimicrobial activity of chimeric Ply187AN-KSH3b in vitro, bacteria (10⁵ CFU) were incubated with chimeric Ply187AN-KSH3b in the presence of calf VH. Following incubation at 37° C. for the desired time, bacterial growth in VH was enumerated by serial dilution plating. To determine the inoculum effect on chimeric Ply187AN-KSH3b treatment, the S. aureus strain RN6390 was grown to logarithmic phase (OD₆₀₀ 0.4-0.6) and 10 fold serial dilutions were made in PBS starting from 1:10 to 1:10,000. These bacterial dilutions were treated with Ply187 (as per MIC) for 1 h at RT and CFU counts were enumerated by dilution plating on TSA plates.

Having shown that chimeric Ply187AN-KSH3b treatment attenuates the clinical symptoms of endophthalmitis and having demonstrated its in vitro antimicrobial activity, we next examined the effect of chimeric Ply187AN-KSH3b treatment on bacterial clearance in mouse eyes. The intravitreal injection of chimeric Ply187AN-KSH3b at 6 h and 12 h post infection drastically reduced bacterial burden in the eyes (FIG. 4A). We observed that on average 1.04×10⁵ CFU/eye were recovered from the EB-treated eyes as compared to 3.6×10³ and 2.9×10⁴ CFU/eye in 6 h and 12 h treatment group, respectively. To rule out the possibility that the observed antimicrobial activity could be due to inherent antimicrobial activity of VH, we tested the staphylolytic activity of chimeric Ply187AN-KSH3b in the presence of VH. As shown in FIG. 4B, the presence of VH does not adversely affect the killing activity of the chimeric Ply187AN-KSH3b as evidenced by the time-dependent reduction in the viable count of S. aureus in treatment groups. In contrast, the bacteria continue to grow in the VH in the control group. To determine whether bacterial concentration influences Ply187AN-KSH3b activity, we performed in vitro inoculum study. To this end, our data showed that there was no reduction in Ply187AN-KSH3b activity with increasing bacterial numbers (FIG. 4C). Altogether, these data indicate that chimeric Ply187AN-KSH3b is an effective therapeutic agent in the inhibition of bacterial growth in the eyes.

### Example 7

### Reduced Inflammatory Responses in Chimeric Ply187AN-KSH3b-Treated Eyes

To measure the inflammatory cytokines, retinal extracts were processed by homogenization in 250 μl of PBS, followed by centrifugation at 12,000×g for 10 min. PBS-injected mice were used as normal controls (PBS C) and EB-treated mice were used as vehicle controls (EB C). The protein concentration of the retinal lysate was determined by the Micro BCA™ protein assay kit (Thermoscientific, Rockford, Ill.). Equal amounts of protein were used to perform the ELISA, per the manufacturer's instructions (BD biosciences, San Diego, Calif. (IL-6, IL1β & TNFα) and R & D systems, Minneapolis, Minn. (MIP2 & KC).

Following euthanasia, the retinas were isolated from the eyes as described by Skeie et al. (2011. J. Vis. Exp. 50:2795) and were digested in Accumax (Millipore, Billerica, Mass.) for 10 min at 37° C. Retinas from two eyes were pooled together to obtain a sufficient number of cells. Following digestion, the retinal tissue was passed through a 23-gauge needle & syringe and filtered through a 40 μm cell strainer (BD Falcon, San Jose, Calif.). The cells were incubated with Fc Block (BD biosciences, San Jose, Calif.) for 30 min, followed by a washing step with PBS containing 0.5% bovine serum albumin (BSA). Cells were then incubated with the phycoerythrin (PE)-Cy5 conjugated CD45, and Ly6G-FITC antibodies (BD biosciences, San Jose, Calif.) for 30 min in the dark. After subsequent washing steps, the cells were acquired on an Accuri C6 flow cytometer (Accuri, Ann Arbor, Mich.), and the data were analyzed using Accuri C6 software (Accuri, Ann Arbor, Mich.).

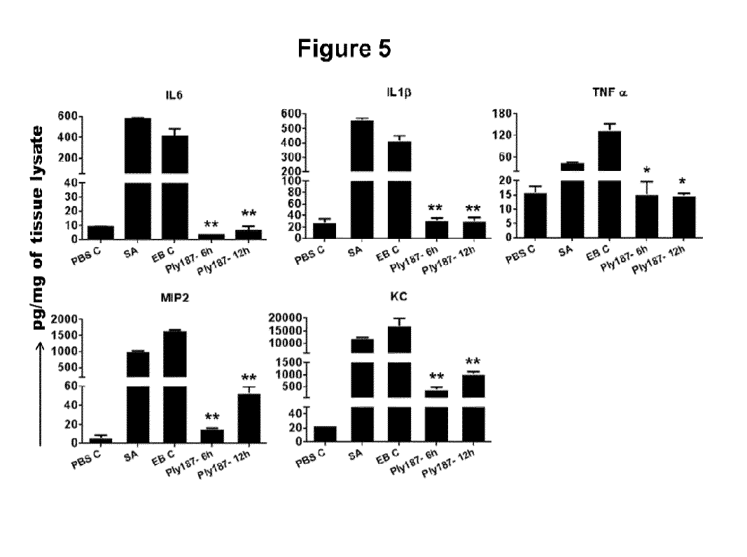

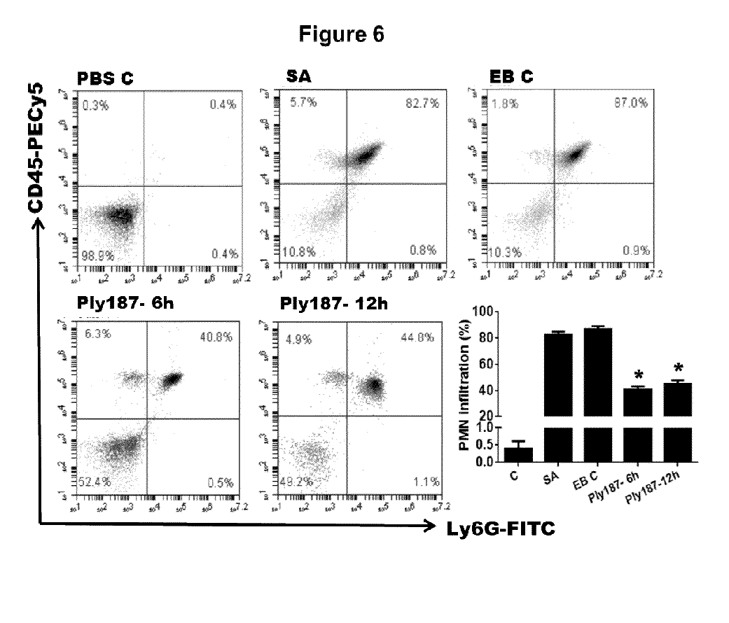

One of the hallmarks of staphylococcal endophthalmitis is the increased levels of inflammatory cytokines and chemokines (Callegan et al. 1999. Infect. Immun. 67:3348-3356). We observed that chimeric Ply187AN-KSH3b treatment significantly suppressed the inflammatory response as evidenced by dramatically reduced levels of IL-6, IL-113, TNFα, MIP2 (CXCL2), and KC (CXCL1) in the Ply187AN-KSH3b-treated eyes as compared to vehicle-treated and S. aureus control eyes (FIG. 5). Both the 6 h and 12 h treatment groups showed a similar reduction in the levels of the assessed cytokines/chemokines with the exception of MIP-2, which showed greater reduction after 6 h vs. 12 h treatment. Similar to the reduction in the levels of inflammatory mediators, our flowcytometry data revealed a ˜50% reduction in neutrophil (PMN) infiltration in eyes treated with chimeric Ply187AN-KSH3b, as compared to vehicle-treated eyes (FIG. 6).

### Example 8

### Chimeric Ply187AN-KSH3b Allows Maintenance of Normal Retinal Function

Scotopic ERG was used to determine retinal function following S. aureus infection and chimeric Ply187AN-KSH3b treatment (6 h post infection). The mice (control, infected and infected+treated) were anesthetized 24 h post Ply187AN-KSH3b treatment, maintained at 37° C. using a heat pad, and the pupils were dilated using 1% tropicamide ophthalmic solution. ERGs were recorded following bilateral mydriasis and at least 4 h of dark adaptation. Silver embedded thread eye electrodes (Ocuscience™ LLC, Kansas City, Mo.) were used to record the ERG. Reference needle electrodes (stainless steel subdermal needle electrodes) were placed in anterior scalp and a ground needle electrode was placed in the tail. ERG responses were acquired using an ERG system (Ocuscience™ LLC, Kansas City, Mo.) and analyzed using ERGVIEW 4.380V. Ganzfeld light stimulus was used to present ten 10 ms flashes, with light intensities, increasing from 0.0001 to 100 cd-s/m². The ERG a-wave was measured as amplitude between the ERG baseline and the first negative peak, and the ERG b-wave was measured as amplitude between the first negative peak and the first positive peak.

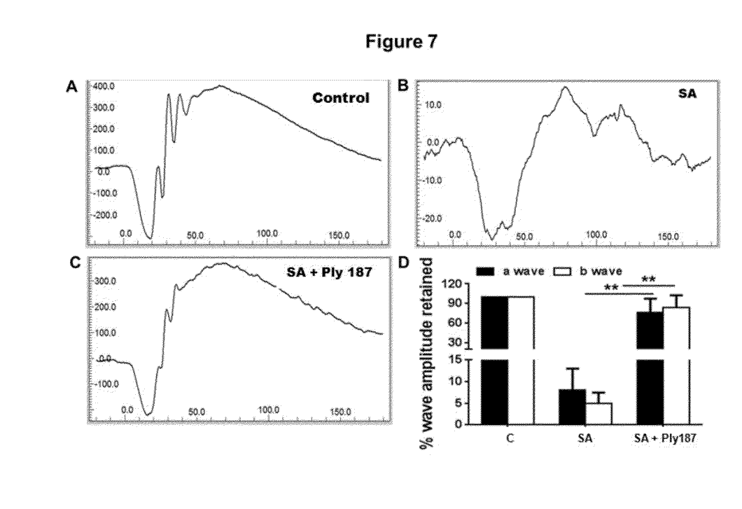

Our previous results demonstrate that chimeric Ply187AN-KSH3b treatment significantly diminished the pathophysiology of staphylococcal endophthalmitis. To determine whether chimeric Ply187AN-KSH3b treatment also preserves retinal function in infected eyes, we determined scotopic ERG responses. The ERG response showed both normal a-waves (the response generated from photoreceptors) and b-waves (the response generated from the inner retina, mostly the bipolar and Muller cells) in the control (uninfected) and S. aureus infected, chimeric Ply187AN-KSH3b-treated eyes. Mice that received intravitreal injections of S. aureus demonstrated the loss of retinal function with a significant decrease in both a-wave (92%) and b-wave (95%) amplitudes (FIG. 7). Remarkably, S. aureus-infected mice treated with chimeric Ply187AN-KSH3b maintained their normal ERG with no significant decline in a- or b-wave amplitudes.

All publications and patents mentioned in this specification are herein incorporated by reference to the same extent as if each individual publication or patent was specifically and individually indicated to be incorporated by reference.

The foregoing description and certain representative embodiments and details of the invention have been presented for purposes of illustration and description of the invention. It is not intended to be exhaustive or to limit the invention to the precise forms disclosed. It will be apparent to practitioners skilled in this art that modifications and variations may be made therein without departing from the scope of the invention.

We claim:

1. A method for treating an individual with eye infection or eye disease caused by staphylococcal strains, including multidrug-resistant (MDR) Staphylococcus aureus, comprising:
administering to said individual in need thereof an effective amount of a composition comprising the chimeric phage endolysin Ply187AN-KSH3b, comprising the complete truncated Ply187AN peptidoglycan hydrolase polypeptide consisting of the cysteine, histidine-dependent amidohydrolases/peptidase (CHAP) endopeptidase domain of endolysin Ply187 together with one or more of the SH3b cell wall binding domain(s) of native LysK, wherein said administration is effective to treat and ameliorate one or more symptoms of said eye infection and/or said eye disease.

2. A method for protecting an individual from developing eye infection or eye disease caused by staphylococcal strains, including multidrug-resistant (MDR) Staphylococcus aureus, comprising:
administering prophylactically to said individual, prior to surgery or intravitreal injections, an effective amount of a composition comprising the chimeric phage endolysin Ply187AN-KSH3b, comprising the complete truncated Ply187AN peptidoglycan hydrolase polypeptide consisting of the cysteine, histidine-dependent amidohydrolases/peptidase (CHAP) endopeptidase domain of endolysin Ply187 together with one or more of the SH3b cell wall binding domain(s) of native LysK, wherein said administration is effective to protect said individual from developing eye infection or eye disease.

3. The method of claim 1 or 2, wherein said Ply187AN-KSH3b has the amino acid sequence of SEQ ID NO:1.

4. The method of claim 1 or 2, wherein said composition is Ply187AN-KSH3b mixed with a pharmacologically acceptable excipient.

5. The method of claim 1, wherein a symptom of the eye infection or eye disease is selected from the group consisting of an increased ocular bacterial burden, an infiltration of neutrophils, an increase in inflammatory cytokines and chemokines, impaired retinal function as measured by ERG, ocular morbidity, retinal folding, corneal opacity, hyopyon, and scarring of the eye.

6. The method of claim 2, wherein said surgery is cataract surgery.

7. The method of claim 1 or 2, wherein said eye disease is endophthalmitis.

8. The method of claim 1, wherein said administration is by a route selected from the group consisting of intraocular injection, intravitreal injection, eye drops, eye gel, eye ointment, spray, emulsion, suspension, contact lenses, polymers, microspheres and implants.

9. A kit, comprising a composition comprising Ply187AN-KSH3b and a pharmacologically acceptable carrier.
